(19) United States
(12) Patent Application Publication
(10) Pub. No.: US 2024/0410685 A1
(43) Pub. Date: Dec. 12, 2024

(54) MONITORING OBJECTS IN AQUEOUS MEDIA USING OPTICAL COHERENCE TOMOGRAPHY

(71) Applicant: ChromoLogic LLC, Monrovia, CA (US)

(72) Inventors: Matthew BREHOVE, Pasadena, CA (US); Claude ROGERS, Pasadena, CA (US); Naresh MENON, La Canada, CA (US); James ALLINGTON, El Monte, CA (US); Rudra MENON, Glendale, CA (US)

(21) Appl. No.: 18/703,297

(22) PCT Filed: Oct. 21, 2022
(86) PCT No.: PCT/US22/47492

§ 371 Date: Apr. 19, 2024

Related U.S. Application Data
(60) Provisional application No. 63/270,465, filed on Oct. 21, 2021.

(51) Int. Cl. G01B 9/02091; G01B 9/02; G01B 9/02055; G01N 21/17; G01N 21/47; G01N 33/50

(52) U.S. Cl. CPC G01B 9/02091; G01B 9/02044; G01B 9/02076; G01N 21/4795; G01N 33/5008; G01N 2021/1787

(57) ABSTRACT

A system for monitoring states of cells in a bioreactor, the system including: an optical coherence tomography system including: a reference arm coupled to a mirror; a sample arm coupled to an optical probe configured to emit an imaging beam into a bioreactor; a light source; and a spectrometer configured to detect recombined light reflected along the reference arm and the sample arm; and a controller comprising a processor and memory, the controller being configured to receive spectrometer data from the optical coherence tomography system and to compute statistics of objects detected by the optical coherence tomography system.

### CROSS-REFERENCE TO RELATED APPLICATION(S)

This application is a U.S. National Phase Patent Application of International Application Number PCT/US2022/047492, filed on Oct. 21, 2022, which claims priority to and the benefit of U.S. Provisional Patent Application No. 63/270,465, filed in the United States Patent and Trademark Office on Oct. 21, 2021, the entire disclosure of each of which is incorporated by reference herein.

### STATEMENT REGARDING FEDERALLY SPONSORED RESEARCH OR DEVELOPMENT

This invention was made with government support under Contract No. W81XWH-19-C-0081 awarded by the Defense Health Agency. The government has certain rights in this invention.

### FIELD

Aspects of embodiments of the present disclosure relate to systems and methods for in-line monitoring of objects in aqueous media using optical coherence tomography.

### BACKGROUND

Biologics and cell therapies are generally more expensive to manufacture than small molecule drugs due, in part, to the time-intensive and resource-intensive quality control processes in operating facilities that comply with Good Laboratory Practices (GLP) and/or Good Manufacturing Processes (GMP). These quality control measures are especially important in cell therapies such as chimeric antigen receptor t-cell therapy (CAR-T), where quality variations often determine the effectiveness of the treatment.

Comparative quality control processes for cell growth typically involve subsampling of the culture, adding a stain such, as trypan blue, to distinguish live from dead cells, and then imaging the cells either manually or through automated image analysis (see, e.g., Butler, M., Spearman, M. & Braasch, K. Monitoring cell growth, viability, and apoptosis. Methods Mol. Biol. 1104, 169-92 (2014)). This process is time and labor intensive and is generally performed once per day, which limits the ability of scientists and technicians to detect problematic cell cultures and to correct or restart such cultures. Additionally, each time the culture is subsampled, some of the culture is lost and the remaining portion of the culture is at increased risk of contamination.

Some cell culture parameters can be measured in-line and non-destructively such as temperature, pH, dissolved O₂, and dissolved CO₂. These can be measured optically with the addition of a pH indicator or fluorescent O₂ or CO₂ chemical probes or they can be measured electrochemically with ion-selective electrodes. While these parameters (e.g., temperature, pH, dissolved O₂, and dissolved CO₂) can indicate problems with cell growth, they cannot be used to quantify the number of cells in solution. In addition, measuring these parameters is not completely non-invasive because they either may require media additives or they may require a probe to be in direct contact with the media, which undercuts the industry's move to disposable bioreactors because the electrochemical probes that contact the media are expensive and must be reused in order to be economical.

Capacitive cell monitoring systems are capable of in-line viable biomass measurements, but also require the capacitive probe to be directly in contact with the cell culture media which is not suitable for all bioreactor types and may require that the probe be sterilized between cell growths.

Raman spectroscopy has shown promise as an optical method for cell quantification, but cell monitoring solutions based on Raman spectroscopy are not currently available due to difficulties in interpreting the data (e.g., converting the Raman spectroscopy measurements into cell counts).

Therefore, both laboratory research and clinical production would benefit from a cell monitoring method that is completely non-invasive and reduces the labor costs associated with monitoring.

The above information disclosed in this Background section is only for enhancement of understanding of the present disclosure, and therefore it may contain information that does not form the prior art that is already known to a person of ordinary skill in the art.

### SUMMARY

Aspects of embodiments of the present disclosure relate to systems and methods for monitoring objects in aqueous media based on optical coherence tomography. One example of objects in aqueous media that may be monitored is cell growths (e.g., a growth in a number of biological cells) in suspended agitated bioreactors.

According to some example embodiments of the present disclosure, a system for monitoring states of cells in a bioreactor, the system including: an optical coherence tomography system including: a reference arm coupled to a mirror; a sample arm coupled to an optical probe configured to emit an imaging beam into a bioreactor; a light source; and a spectrometer configured to detect recombined light reflected along the reference arm and the sample arm; and a controller including a processor and memory, the controller being configured to receive spectrometer data from the optical coherence tomography system and to compute statistics of objects detected by the optical coherence tomography system.

According to some example embodiments, the memory stores instructions that, when executed by the processor, cause the processor to: capture the spectrometer data from the optical coherence tomography system over a capture period; compute a plurality of 1D images from the spectrometer data, each of the 1D images representing depths of one or more reflective surfaces in the bioreactor from the optical probe along an axis of the imaging beam; generate a plurality of cell monitoring optical coherence tomography (CM-OCT) frames including a plurality of 1D images collected over the capture period; and compute statistics of the objects detected in the CM-OCT frames.

According to some example embodiments, the capture period is about 65 ms.

According to some example embodiments, the memory stores instructions that, when executed by the processor, cause the processor to compute the statistics of the objects detected in the CM-OCT frames by: detecting and removing a window of the bioreactor from the CM-OCT frames; detecting the objects in the CM-OCT frames; computing size and backscatter intensity parameters of each of the objects in the CM-OCT frames, the size parameters including a height in a depth dimension and a width in a time dimension of the CM-OCT frames; and computing the statistics of the objects based on the size and backscatter intensity parameters of the objects.

According to some example embodiments, computing the size and backscatter intensity parameters of each of the objects includes a multi-dimensional regression analysis to the object.

According to some example embodiments, the statistics of the objects include a concentration of cells in the bioreactor.

According to some example embodiments, the plurality of 1D images computed are analyzed using machine learning methods to identify live and dead cells in the bioreactor.

According to some example embodiments, the memory stores instructions that, when executed by the processor, further cause the processor to determine a set of operating parameter ranges for the statistics.

According to some example embodiments, the statistics of the objects include a viability metric of cells in the bioreactor based on the backscatter intensity parameters of the objects.

According to some example embodiments, the light source includes a super-luminescent diode.

According to some example embodiments, the optical probe includes a 10× objective.

According to some example embodiments, the light source has a bandwidth of between 150 and 200 nm.

According to some example embodiments, the optical probe has a numerical aperture of about 0.064.

According to some example embodiments, the optical probe includes an achromatic lens.

According to some example embodiments, the optical probe is coupled to the bioreactor such that the imaging beam is substantially perpendicular to a wall of the bioreactor.

According to some example embodiments, the reference arm includes a variable optical attenuator.

According to some example embodiments of the present disclosure, in a method for monitoring states of cells in a bioreactor using an optical coherence tomography system including a processor and memory, the method including: capturing, by the processor, spectrometer data from the optical coherence tomography system over a capture period; computing, by the processor, a plurality of 1D images from the spectrometer data, each of the 1D images representing depths of one or more reflective surfaces in the bioreactor from an optical probe system along an axis of an imaging beam; generating, by the processor, a plurality of cell monitoring optical coherence tomography (CM-OCT) frames including a plurality of 1D images collected over the capture period; and computing, by the processor, statistics of objects detected in the CM-OCT frames.

According to some example embodiments, the capture period is between 10 and 100 ms.

According to some example embodiments, the method of computing the statistics of the objects detected in the CM-OCT frames includes: detecting and removing a window of the bioreactor from the CM-OCT frames; detecting the objects in the CM-OCT frames; computing size and backscatter intensity parameters of each of the objects in the CM-OCT frames, the size parameters including a height in a depth dimension and a width in a time dimension of the CM-OCT frames; and computing the statistics of the objects based on the size and backscatter intensity parameters of the objects.

According to some example embodiments, the method for computing the size and backscatter intensity parameters of each of the objects further includes a multi-dimensional regression analysis to the object.

According to some example embodiments, the statistics of the objects include a concentration of cells in the bioreactor.

According to some example embodiments, the plurality of 1D images computed are analyzed using machine learning methods to identify live and dead cells in the bioreactor.

According to some example embodiments, the statistics of the objects include a viability metric of cells in the bioreactor based on the backscatter intensity parameters of the objects.

According to some example embodiments, a reference arm of the optical coherence tomography system includes an optical arm including a variable optical attenuator.

According to some example embodiments of the present disclosure, a system for monitoring states of cells in a plurality of bioreactors, the system including: an optical coherence tomography system including: a reference arm coupled to a mirror; a sample arm coupled to a first optical probe configured to emit a first imaging beam into a first bioreactor; the sample arm coupled to a second optical probe configured to emit a first imaging beam into a second bioreactor; an optical switch coupled to the sample arm configured to switch between the first optical probe and the second optical probe; a light source; and a spectrometer configured to detect recombined light reflected along the reference arm and the sample arm; and a controller including a processor and memory, the controller being configured to receive spectrometer data from the optical coherence tomography system and to compute statistics of objects detected by the optical coherence tomography system.

### BRIEF DESCRIPTION OF THE DRAWINGS

The accompanying drawings, together with the specification, illustrate exemplary embodiments of the present invention, and, together with the description, serve to explain the principles of the present invention.

FIG. 1A shows a schematic diagram of a cell monitoring optical coherence tomography (CM-OCT) system with an optical probe according to one embodiment of the present disclosure.

FIG. 1B is a schematic diagram of an alternative probe attachment to the same system containing a 10× objective according to one embodiment of the present disclosure.

FIG. 1C is an image of CM-OCT data collected over 65 ms according to one embodiment of the present disclosure.

FIG. 1D is a photograph of a 10× objective optical probe imaging a bioreactor according to one embodiment of the present disclosure.

FIG. 1E is a cross sectional diagram of a custom optical probe according to one embodiment of the present disclosure.

FIG. 1F includes a diagram and a photograph of a probe mount according to one embodiment of the present disclosure.

FIG. 1G includes a diagram and a photograph of a probe mount used with a rotating wheel bioreactor manufactured by PBS Biotech Inc. according to one embodiment of the present disclosure.

FIG. 1H is a flowchart of a method for performing cell monitoring using a CM-OCT system according to one embodiment of the present disclosure.

FIG. 1I is a flowchart of a method for performing object detection analysis on CM-OCT frames according to one embodiment of the present disclosure.

FIG. 2A is a graph illustrating the number of objects detected per second by a CM-OCT system according to one embodiment of the present disclosure in comparison with cell concentration as measured by a hemocytometer for two different types of cells (CHO and UT7).

FIG. 2B shows graphs of the distribution of detected sizes of cells measured by brightfield image analysis in comparison with a CM-OCT system according to one embodiment of the present disclosure.

FIG. 3A depicts a comparison of cell viability measures made by a comparative XTT assay absorbance versus cell backscatter intensity measurements made by a CM-OCT system according to one embodiment of the present disclosure.

FIG. 3B depicts cell viability measures based on average cell backscatter intensity measured by a CM-OCT system according to one embodiment of the present disclosure.

FIG. 3C depicts cell viability measures based on an XTT assay absorbance according to one embodiment of the present disclosure.

FIGS. 4A-D shows data from culture of cells grown over 170 hours in a bioreactor that was monitored by both CM-OCT and a comparative Innovatis Cedex® HiRes system (Cedex) according to one embodiment of the present disclosure.

FIG. 4A shows cell concentration measurements by CM-OCT and the Cedex system according to one embodiment of the present disclosure.

FIG. 4B shows the viability and relative intensity of backscattered light as measured by the CM-OCT according to one embodiment of the present disclosure.

FIG. 4C shows CM-OCT and Cedex measurements of cell concentration taken with cultures with >90% viability in each of the three independent growths according to one embodiment of the present disclosure.

FIG. 4D shows viability data from three independent growths as measured by Cedex vs relative intensity measured by CM-OCT according to one embodiment of the present disclosure.

FIGS. 5A-5C respectively depict the distribution of detected object sizes as measured by the comparative Cedex analyzer and a CM-OCT system according to one embodiment of the present disclosure at the beginning of growth, after 100 hours (as cells are beginning to die), and after 170 hours (when cell viability is low), respectively.

FIGS. 5D-5F depict Cedex images used to measure the object size distribution at the beginning of growth, after 100 hours (as cells are beginning to die), and after 170 hours (when cell viability is low), respectively.

### DETAILED DESCRIPTION

In the following detailed description, only certain exemplary embodiments of the present invention are shown and described, by way of illustration. As those skilled in the art would recognize, the invention may be embodied in many different forms and should not be construed as being limited to the embodiments set forth herein.

Aspects of embodiments of the present disclosure relate to systems and methods for monitoring objects in aqueous media based on optical coherence tomography (OCT). Some aspects of embodiments of the present disclosure will be presented herein for the specific case of monitoring the growth of biological cells suspended in an aqueous growth medium in a bioreactor, but embodiments of the present disclosure are not limited thereto. For example, embodiments may be used to monitor the states of various other types of objects suspended in an aqueous medium, such as bacteria or polystyrene beads, where the object and the aqueous medium have a different index of refraction. Some example aqueous media include Luria-Bertani (LB), Minimum Essential Media (MEM), Dulbecco's Modified Eagle Medium (DMEM), but is not limited thereto. Therefore, embodiments of the present disclosure may be used in, for example, various industrial manufacturing processes (e.g., manufacturing of chemical and biological products). Embodiments of the present disclosure are able to monitor growing cell cultures in bioreactors using a non-invasive optical probe mounted outside of the bioreactor. This enables the user to monitor the culture without opening the bioreactor to remove samples and thereby reduce the risk of contamination. As such, embodiments of the present disclosure provide systems and methods that are in-line, completely non-invasive, and that can operate on any bioreactor with a window or transparent wall. While some comparative techniques of performing OCT raster an imaging beam in one dimension to form a two-dimensional (2D) image or raster the imaging beam in two dimensions to form a three-dimensional (3D) image of the cells in culture, in some embodiments of the present disclosure, a fixed imaging beam is used (e.g., without rastering the beam) and, instead, the motion of the aqueous media through the bioreactor is used to move the cells across the beam. Since the system's measurements require neither consumable materials nor interaction with the user, they can be run continuously and in real time in order to give a more fine-grained picture of how the cells are developing. More broadly, embodiments of the present disclosure may be applied to perform in-line monitoring of objects in other biochemical or chemical processes (e.g., industrial manufacturing processes), where the objects of interest have an index of refraction that is different from that of the aqueous medium.

Experiments have shown that embodiments of the present disclosure are able to quantify the cell concentration as well as the average cell size and viability by analyzing the OCT signal over time. Comparing the measurements with comparative trypan blue hemocytometer measurements, automated cell counter measurements (e.g., Innovatis Cedex HiRes), and XTT viability assays and found strong correlations on all three metrics (p>0.84) in growing cell cultures, thereby validating the technique of embodiments of the present disclosure. Embodiments of the present disclosure provide methods for cell monitoring that can adapt to different bioreactor form factors and that can reduce the labor costs associated with cell growth monitoring.

Generally, optical coherence tomography (OCT) is an interferometric technique that can acquire micron-resolution, cross-sectional images of biological samples (see, e.g., Schmitt, J. M. Optical Coherence Tomography (OCT): a review. IEEE J. Sel. Top. Quantum Electron. 5, 1205-1215 (1999)). According to some embodiments of the present disclosure, a cell monitoring optical coherence tomography (CM-OCT) system is based on Spectral-Domain (SD-OCT) which uses a wide bandwidth light source to perform low-coherence Michelson interferometry and detects the re-combined reference and sample arms using a spectrometer. The bandwidth of the light source used determines the axial resolution according to l=0.44λ₀²/Δλ where Δλ is the bandwidth and λ₀ is the center wavelength (Schmitt, J. M. Optical Coherence Tomography (OCT): a review. IEEE J. Sel. Top. Quantum Electron. 5, 1205-1215 (1999)). The minimum required bandwidth for 2 μm resolution with an 890 nm center wavelength is 180 nm.

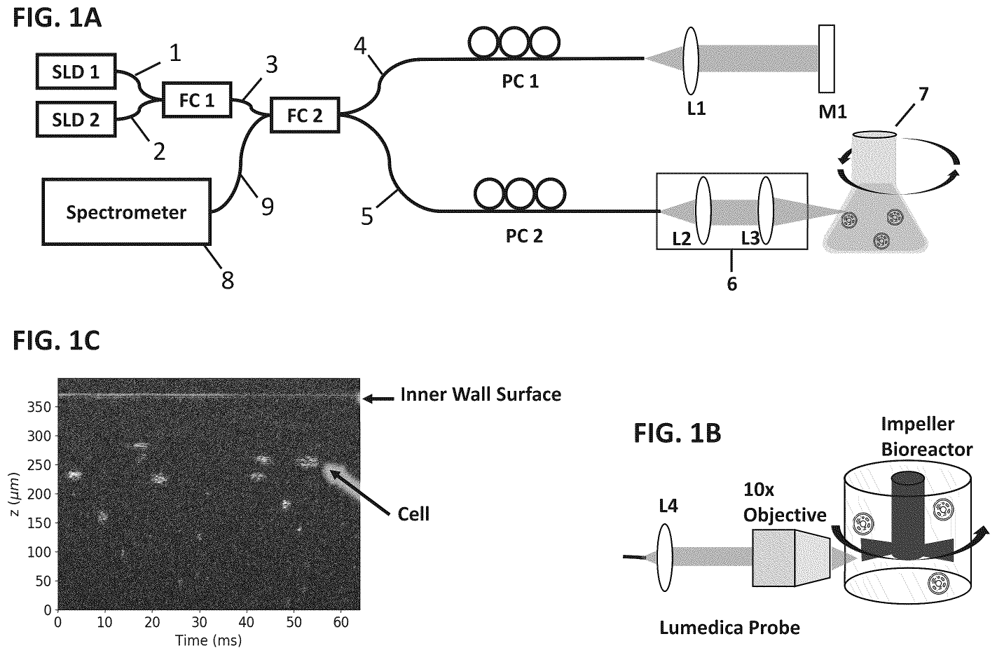

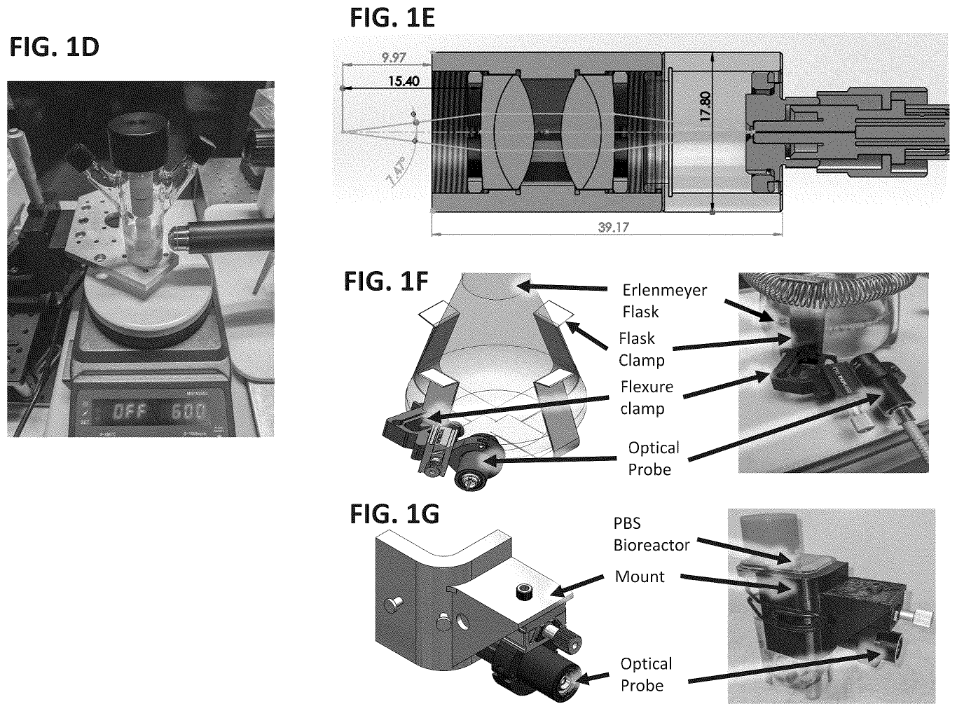

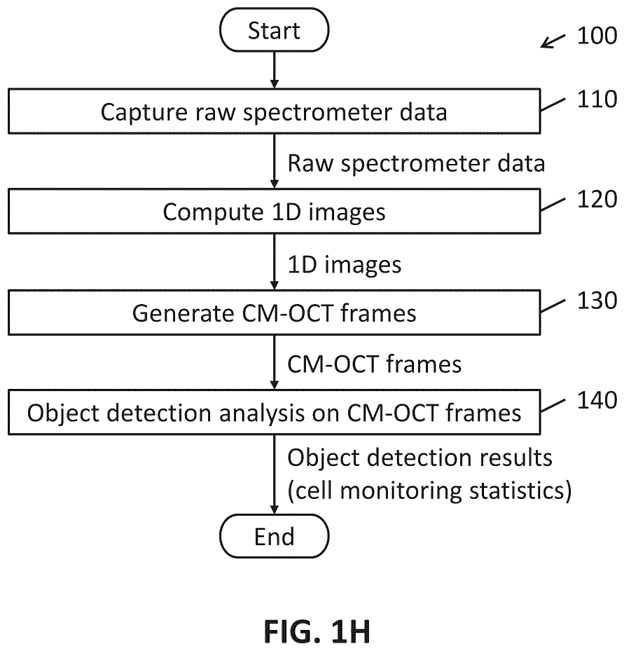

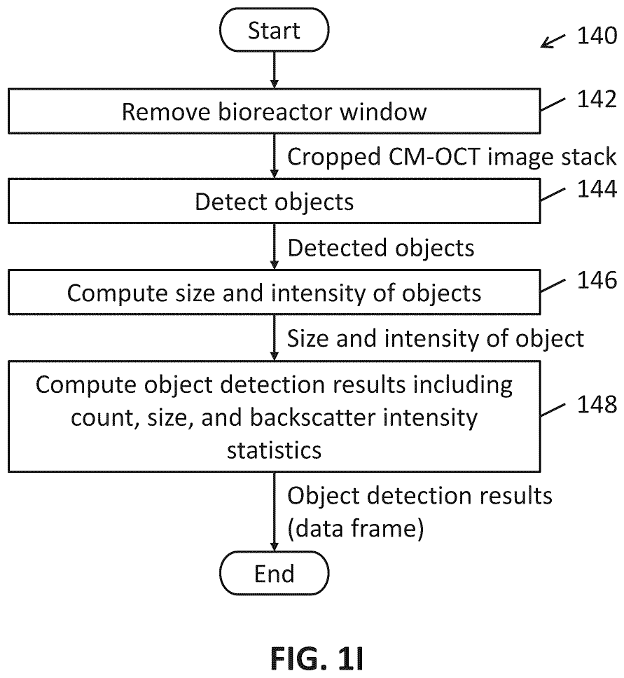

FIG. 1A shows a schematic diagram of a cell monitoring optical coherence tomography (CM-OCT) system with an optical probe according to one embodiment of the present disclosure.

In the arrangement shown in FIG. 1A, light emitted from light sources SLD 1 and SLD 2 (e.g., super-luminescent diodes) are supplied via fiber optic cables 1 and 2. The fiber optic cables 1 and 2 may be combined by a first fiber coupler FC 1 into fiber optic cable 3. Fiber optic cable 3 may then be split by a second fiber coupler FC 2 into a reference arm fiber optic cable 4 and a sample arm fiber optic cable 5. The reference arm fiber optic cable 4 and sample arm fiber optic cable 5 may include polarization controllers PC 1 and PC 2 respectively to reduce any mismatch in polarization between the reference arm optic cable 4 and the sample arm optic cable 5. Light beam emitted from the reference arm optical cable 4 may be expanded by a lens L1 and the light beam may be reflected by a mirror M1 so that the length of the light beam from the reference arm optical cable 4 may be adjusted.

Light beam emitted from sample arm optical cable 5 may pass through an optical probe 6 and into a bioreactor 7 through a transparent side of the bioreactor (e.g., shake flask). The bioreactor 7 may include objects (e.g., biological cells) in an aqueous medium. In some embodiments, for example as shown in FIG. 1A, the optical probe 6 may include two lenses L2 and L3. In some embodiments, the optical probe 6 may include a 10× objective optical probe such as in the embodiment illustrated in FIG. 1B. As illustrated in FIG. 1B, the 10× objective optical probe may be placed adjacent to a transparent side of the bioreactor (e.g., impeller bioreactor). Referring back to FIG. 1A, the light beam from the reference arm optical cable 4 may be reflected by the mirror M1 and the light beam from the sample arm optical cable 5 may be reflected by objects in the bioreactor 7. The two reflected light beams from the reference arm optical cable 4 and the sample arm optical cable 5 may be recombined by the second fiber coupler FC 2 and into a spectrometer 8 via fiber optic cable 9. The recombined beam is then detected on the spectrometer 8.

The OCT optical setup described above in FIG. 1 uses spectral domain OCT (SD-OCT) in which all wavelengths are used simultaneously throughout the optics before finally being separated and detected using a spectrometer, but other embodiments may use swept source OCT (SS-OCT) in which a variable wavelength laser is used in place of a broad-spectrum light source. In SS-OCT the wavelengths are separated temporally and detected using a high-speed photodetector. Other aspects of the system including the reference arm, sample arm, fiber couplers and data analysis would be equivalent.

In the reference arm some embodiments may include a variable optical attenuator (VOA) that could allow the power in the reference arm to be adjusted independently of the sample arm.

Using the spectrometer 8, the system performs a Fourier transform to calculate a 1-dimensional (1D) image of the reflective surfaces along the beam path (referred to herein as the z-axis or “depth”). Each frame from the spectrometer is processed to form a 1D image of the sample. FIG. 1C shows an image of 512 frames taken over 65 ms according to an embodiment of the present disclosure. The inner wall of the bioreactor is visible as a bright horizontal line and cells are visible as short streaks as they pass through the imaging beam.

Spectral-domain optical coherence tomography (SD-OCT) systems such as those used in some embodiments have improved signal-to-noise ratios over comparative time-domain (TD) OCT systems. As noted above, comparative OCT systems generally raster the imaging beam over one dimension (e.g., an x-axis perpendicular to the z-axis of the beam) to produce a 2D image of a sample or raster the imaging beam over two dimensions (e.g., x- and y-axes perpendicular to the z-axis of the beam) to produce a 3D image of a sample. However, this method of imaging is not feasible for large-scale cell cultures in which the cells are suspended in moving media and therefore the cells cannot remain fixed (or static) while the beam rasters or sweeps over its field of view. In contrast, embodiments of the present disclosure use a one-dimensional OCT scan, which images cells along the direction of the imaging beam which passes perpendicularly through the bioreactor window (see, e.g., FIG. 1A). As the media moves, cells cross this beam and, over time, the system forms a distance-vs-time image (FIG. 1C) which is then analyzed to determine cell concentration, size and viability. In this way, CM-OCT takes advantage of cell movement in order to quantify a representative sample of the cultured cells by effectively capturing a 2D image or set of images of the sample as the same moves across the fixed beam (e.g., rather than moving the beam across a fixed sample, as performed by comparative OCT systems).

While FIG. 1A shows a system where a single optical probe is connected to the super-luminescent diodes and the spectrometer, embodiments of the present disclosure are not limited thereto. For example, some aspects of embodiments relate to multi-probe systems that may include multiple optical probes imaging different samples and may be connected to the same spectrometer and the same wide bandwidth light source using an optical switch in the sample arm. This may enable the system to switch between different optical probes that share a same spectrometer and same wide bandwidth light source, such that multiple samples can be monitored on shared equipment, and thereby reducing the overall cost of the system. For the sake of convenience and clarity, embodiments of the present disclosure will be presented herein using embodiments of a single optical probe and without such an optical switch, without limitation to such embodiments.

In one particular example embodiment of a CM-OCT system of the present disclosure, the OCT system may include two super-luminescent diodes (SLD's) which give the system a center wavelength of about 890 nm and a total bandwidth of about 185 nm. These wavelengths result in a theoretical axial resolution (e.g., along a direction of the beam) of about 1.85 μm. The system may also include a 2048-pixel spectrometer, which can be sensitive to about 774-991 nm light. In some embodiments, the OCT system is configured to acquire axial scans (e.g., A-scans) at a rate of about 6.5 kHz which may result in about 12.7 z-vs-time images per second (or frames per second).

Some aspects of embodiments relate to a CM-OCT system that include an optical probe with a 10× objective. For example, an OCT probe may include a 10× objective as shown in FIGS. 1B and 1D, which may be used to image cell growth cultures in an impeller bioreactor. FIG. 1D is a photograph of a 10× objective optical probe imaging a bioreactor, which sits on an x-y-z stage above a magnetic stir plate.

FIG. 1E is a cross sectional diagram of a custom optical probe according to one embodiment of the present disclosure. As noted above, some aspects of embodiments of the present disclosure relate to a custom OCT probe that may be small enough to be attached to a flask (e.g., shake flasks), such as in the embodiments illustrated in FIGS. 1A and 1E. According to these embodiments, the OCT probe may omit the x-y scanning hardware used for 3D imaging in a comparative scanning OCT probe. In the OCT probe shown in FIG. 1E, a beam path may begin with the fiber on the right side of the diagram, which passes through two achromatic lenses and focuses the beam at a working distance of about 15.4 mm. In various embodiments of the present disclosure, the working distance of the probe may be larger than a thickness of a side of the bioreactor (e.g., a bioreactor wall or window) in order for the focal point to extend a sufficient depth (e.g., at least 200 μm when imaging some cell growth cultures) into the media to image objects in the bioreactor. Bioreactor windows can range from hundreds of microns to several millimeters. With the OCT probe, as with any optical objective, there may be a tradeoff with larger working distances being associated with smaller numerical apertures. Unlike traditional microscopy, very high numerical apertures (NA) are not desirable for these applications because this decreases the depth of field (e.g., the distance over which objects are in focus) and therefore decreases the number of detected objects. The spatial resolution is determined by the spectrometer resolution and the light source bandwidth so there is no decrease in resolution that might be typically associated with low NA objectives. Therefore, the NA only needs to be sufficiently high to capture enough reflected light for the object to be detected. The embodiment of the probe described herein has a numerical aperture of about 0.064, which can yield about a 15.4 mm working distance and may be compatible with a wide range of bioreactors. The optical probe in this embodiment may use achromatic lenses so that all wavelengths can be focused at the same location which reduces the need to correct for chromatic aberration in software.

FIG. 1F includes a diagram and a photograph of a probe mount according to one embodiment of the present disclosure. Some aspects further relate to a probe mount capable of attaching a probe to a flask (e.g., shake flask). The probe mount may include a custom machined aluminum clamp, a linear stage (e.g, to adjust the distance between the probe and the flask), and an attachment point for the optical probe. The lightweight aluminum clamp may attach to one of the arms of the flask clamp and can provide a rigidity that can reduce or minimize measurement noise.

FIG. 1G includes a diagram and a photograph of a probe mount used with a bioreactor developed by PBS Biotech Inc according to one embodiment of the present disclosure. Some aspects further relate to a probe mount capable of attaching a probe to a PBS bioreactor. The probe mount may attach to a side of the PBS bioreactor.

To align the probe, the probe angle may be adjusted such that the beam is substantially perpendicular to the bioreactor wall. Then the probe's z-position may be adjusted using the linear stage until the inner surface of the bioreactor is visible (e.g., the inner surface of the transparent flask or the inner surface of a transparent window of the bioreactor). Then the reference arm and the probe position may be alternately adjusted until the cells near the inner surface of the bioreactor are in focus. This process can assure that both the focal plane of the optics and the reference plane are substantially aligned just inside the inner wall, where the physical interactions with the surface of the bioreactor can generally cause the cells to move more slowly than deeper within the bioreactor. This adjustment may be performed each time a new flask or bioreactor is used for imaging because the glass thickness varies between vessels and may also vary between different parts of the same vessel or different parts of a window of a bioreactor.

FIG. 1H is a flowchart of a method for performing cell monitoring using a CM-OCT system according to one embodiment of the present disclosure. The method may be performed by a controller system including a processor and memory. The controller system may be connected to the spectrometer to receive data from the spectrometer and may also be connected to control other portions of the CM-OCT system, such as the emission of light by the super-luminescent diodes SLD 1 and SLD 2 and/or to control the position of the actuated mirror M1.

Referring to FIG. 1D, in operation 110 of FIG. 1H, the controller system captures a plurality of frames of spectrometer data (e.g., raw spectrometer data output by the spectrometer) using a CM-OCT according to embodiments of the present disclosure with an optical probe adjusted to image a cell culture in a bioreactor through a transparent window or transparent wall of the bioreactor (as described above).

In operation 120, the controller system processes the raw spectrometer data. This may include background subtraction, dispersion correction, and the inverse Fourier transform. The background to be subtracted may be determined by averaging the spectrum over a large number of scans (e.g., 512 scans) and the dispersion correction may be performed by multiplying each element of the spectrum with a complex phase factor ϕ before taking the inverse Fourier transform of the data, where the complex phase factor ϕ may be defined as:

$$ϕ = - i ( {bn}^{2} + {cn}^{3} ) \qquad ( 1 )$$

where b and c are manually optimized correction factors found, in some embodiments, to be −250 and 15, respectively. The result of the inverse Fourier transform is a 1 D image of the sample along the imaging axis. In some embodiments, 512 such images can be taken to generate one CM-OCT image frame (as such, the scanning rate of 6.5 kHz generates 6,500 images per second, and using 512 images per CM-OCT image frame results in the above-noted rate of 12.7 CM-OCT frames/second). This frame represents a z-vs-time matrix of backscatter intensity as shown, for example in FIG. 1C. This image may then be log transformed and scaled according to equation (2).

$${Image}_{transform} = 65536 \times \frac{20 \times {log}_{10} ( Image ) - 40}{95 - 40} \qquad ( 2 )$$

In one embodiment, the wavelength spacing of the spectrometer resulted in a pixel size in the z-dimension is about 1.3 μm in air, and about 1.0 μm in water, after correcting for the index of refraction in water.

Each frame of 1D data represents the distances (or depths) of reflective surfaces within the working distance of the optical probe (e.g., about 15.4 mm) along the axis of the beam. As noted above, as one example, these images or axial scans (A-scans) may be captured at a rate of about 6.5 kHz. In some embodiments, 512 scans may be combined for each image, which may result in about 12.7 z-vs-time images per second (or CM-OCT frames per second).

In operation 130, the controller system combines (e.g., concatenates) the 1D frames captured over a capture period to form a 2D image. As noted above, FIG. 1C provides an example of such a 2D image generated by combining 1 D frames of CM-OCT data over a capture period of about 65 ms.

In operation 140, the controller system performs object detection analysis on the collected images. In some embodiments, a stack of approximately 200 such images may be collected before object detection analysis is performed.

FIG. 1I is a flowchart of a method for performing object detection analysis on CM-OCT frames according to one embodiment of the present disclosure.

In some embodiments, to perform object identification, the first step is to locate and remove the inner bioreactor window surface in operation 142. Assuming proper alignment of the optical probe, as described above, the inner surface of the window or transparent wall of the bioreactor may be the brightest at the closest peak to the optical probe. As such, in some embodiments, the system controller may calculate the mean pixel value along the z-dimension (mean_z), and then can identify the z-location with the highest average intensity, such as by using the find_peaks( ) function in the scipy.signal python package. In some embodiments, if the window is not detected within a bounded range from the top of the image (e.g., within about 175-275 pixels), then the system controller may provide an error message telling the user that the optical probe is out of alignment. The image stack may then be cropped along the z-axis to z-pixels between about 20 and 320 pixels below the surface location. This may remove the window from the analysis and can assure that only cells within a fixed distance from the bioreactor wall can be analyzed. In some embodiments, the mean_z vector may be used once more to perform background subtraction on the cropped image.

In operation 144, the controller system detects objects in the CM-OCT image stack. In some embodiments, connected component labeling is used to detect the presence of objects (see, e.g., He, L., Chao, Y., Suzuki, K. & Wu, K. Fast connected-component labeling. Pattern Recognit. 42, 1977-1987 (2009).). For example, some embodiments may use the SimpleBlobDetector from the OpenCV library to locate the object using a series of thresholds to create a series of identified objects. Based on the expected sizes of cells and the resolution of the CM-OCT system, objects within a particular range of a pixel area may be counted (e.g., between 50 and 200,000 pixels in area). In some embodiments, before feeding each frame to SimpleBlobDetector, the frame may be filtered with a 3×3-pixel uniform filter to smooth the image and reduce noise.

In operation 146, the controller system measures the size and intensity of the detected objects. In some embodiments, multi-dimensional regression analysis (e.g., fitting a multi-dimensional Gaussian distribution) may be used to find the size and intensity values. To perform this analysis, the boundaries of each object are identified. In some embodiments, the boundaries may be identified by creating a binary image for each detected object with a threshold that is substantially halfway between the background and the pixel value at the center of the object. The binary image may be then used to, again, identify the location of the object and its extent in the z-axis (e.g., the depth axis) and in time. The image may be then cropped to contain the extent of the cell and a 10-pixel border around the boundaries of the cell. This cropped image is then processed with a multi-dimensional regression analysis from which three parameters may be extracted: the object's height in z, the object's width in time (σₜ), and the object's backscatter intensity. These parameters along with the object's location in z-axis and time are compiled in operation 148 into a data frame, which may further include object counts based on the number of objects detected in operation 144.

The object detection results (e.g., statistics) may also include a cell concentration. The cell concentration may be proportional to the number of objects detected in the CM-OCT image stack, which are in the target cell size range (e.g., about 16 μm to 21 μm). The constant of proportionally may be determined at the beginning of the growth by taking the ratio of the user-supplied initial cell concentration and the initial observed object counts.

In some embodiments, the object detection statistics may be combined with measurements of other components in the bioreactor to provide a holistic measurement of cell culture health. Some of these other measurements may include pH, glucose concentration, oxygen levels, or CO₂ measurements. By tracking the behavior of the object detection statistics along with these other measurements over repeated successful growths in identical conditions, a set of operating parameter ranges could be defined for each measurement type at each timepoint in the growth. These operating parameters could then be used to define a multi-dimensional envelope of acceptable operating parameters. The system could detect significant drift outside of this envelope and alert the users of potential problems with the growth.

In some embodiments, machine learning methods may be used to analyze the collected images to distinguish live cell from dead cells in the bioreactor. In some embodiments, a labeled dataset is produced by manually labeling regions of images as depicting live cells, dead cells, and no cells (e.g., using bounding boxes or labeling regions of pixels). The labeled dataset may then be divided into a training dataset and a test dataset, where the training dataset is used to train (or retrain) a neural network (e.g., a convolutional neural network or CNN such as Mask R-CNN as described in He, Kaiming, et al. “Mask R-CNN.” Proceedings of the IEEE International Conference on Computer Vision. 2017.), using backpropagation and gradient descent, to compute segmentation maps (e.g., semantic segmentation or instance segmentation) that classify regions of the input images (e.g., based on a confidence that those regions depict live cells, dead cells, or no cells).

### Example 1

To test the performance of a CM-OCT system according to some embodiments of the present disclosure, a CM-OCT system was set up to measure the number of objects that passed through the imaging beam every second (objects per second or OPS). In particular, as noted above, this quantity was hypothesized to be proportional to the cell concentration. In order to test this association, a dilution series using UT7-EPO erythroleukemic cells (UT7) and Chinese hamster ovary (CHO) cells was imaged in a small impeller bioreactor (Wheaton Celstir Double Sidearm Spinner Flask, 38 mm, 25 mL 356873). The dilutions were performed in triplicate and each imaged for 40 seconds each.

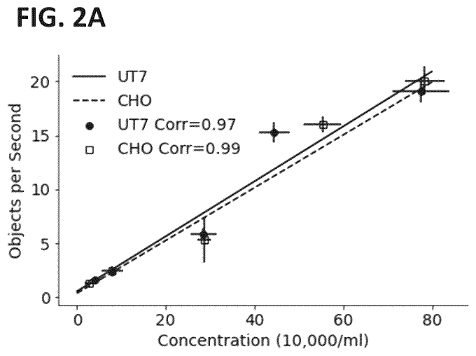

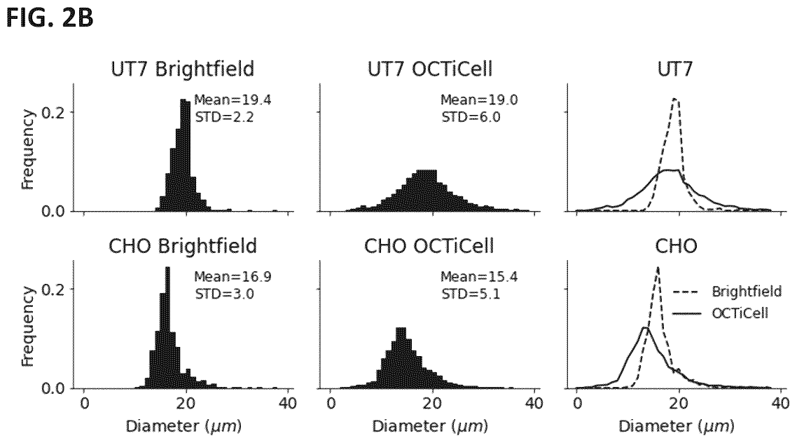

FIG. 2A depicts the number of objects detected per second by a CM-OCT system according to one embodiment of the present disclosure in comparison with cell concentration as measured by a hemocytometer for two different types of cells. In particular, FIG. 2A shows objects detected per second (OPS) in a CM-OCT system according to embodiments of the present disclosure vs cell concentration as measured by a hemocytometer for both UT7 cells and CHO cells. Dashed lines in FIG. 2A show linear fit to the data. The results shown in FIG. 2A show a strong correlation between the detected OPS and the concentration as measured by hemocytometer for both CHO cells and UT7 cells (ρ=0.97 UT7, 0.99 CHO). Additionally, the difference between the OPS/(ml⁻¹) ratio is not significant. Objects per second observed for both UT7 cells and CHO cells were similar at similar concentrations, indicating that CM-OCT would likely not need to adjust any of the analysis parameters when switching between different cell types.

### Example 2

To further investigate this data, the distribution of cell size measured by CM-OCT according to embodiments of the present disclosure was compared against the distribution as measured by brightfield microscopy. FIG. 2B presents graphs of the distribution of detected sizes of cells measured by brightfield image analysis in comparison with a CM-OCT system according to one embodiment of the present disclosure. The brightfield images of the cells were taken immediately after imaging in the bioreactor and the cells were not adhered to the surface. Cell sizes in the images were measured manually using the ImageJ image processing program and the mean cell sizes found in the brightfield images and CM-OCT differed by about 0.4 μm (for UT7 cells) and about 1.5 μm (for CHO cells), which indicates that CM-OCT's measurement of size has relatively low systematic error, and also that this error may vary slightly by cell type, as shown in FIG. 2B.

To estimate the non-systematic error of a CM-OCT system according to embodiments of the present disclosure, the size distributions for both brightfield and CM-OCT data can be approximated as Gaussian distributions. In this approximation, any Gaussian distributed error in the CM-OCT measurement would increase the standard deviation of the CM-OCT measurements according to:

$${σ}_{total}^{2} = {σ}_{original}^{2} + {σ}_{measurement error}^{2}$$

This implies an unbiased measurement error of each cell of about 5.58 μm (for UT7 cells) and about 4.21 μm (for CHO cells).

### Example 3

One important cell culturing metric is the fraction of cells in the culture which are viable. We hypothesized that morphological changes such as blebbing that take place during apoptosis would be detected by OCT as an increase in the intensity of the backscattered light which is detected in the OCT image. These morphological changes make dead CHO cells identifiable in traditional light microscopy because blebbing gives the cell more internal surfaces to reflect the imaging beam and therefore yield a brighter CM-OCT signal.

To test this hypothesis, hydrogen peroxide (H₂O₂) was added to CHO cell cultures at varying concentrations and the viability of the cell cultures was measured by an XTT assay and CM-OCT backscatter intensity. The XTT assay is a standard viability test for growing cell cultures and measures the change in absorbance of 475 nm light with higher absorbance indicating higher viability. The assay uses a tetrazolium dye which changes color when it is reduced as a result of cell metabolism (see, e.g., Kamiloglu, S., Sari, G., Ozdal, T. & Capanoglu, E. Guidelines for cell viability assays. Food Front. 1, 332-349 (2020).).

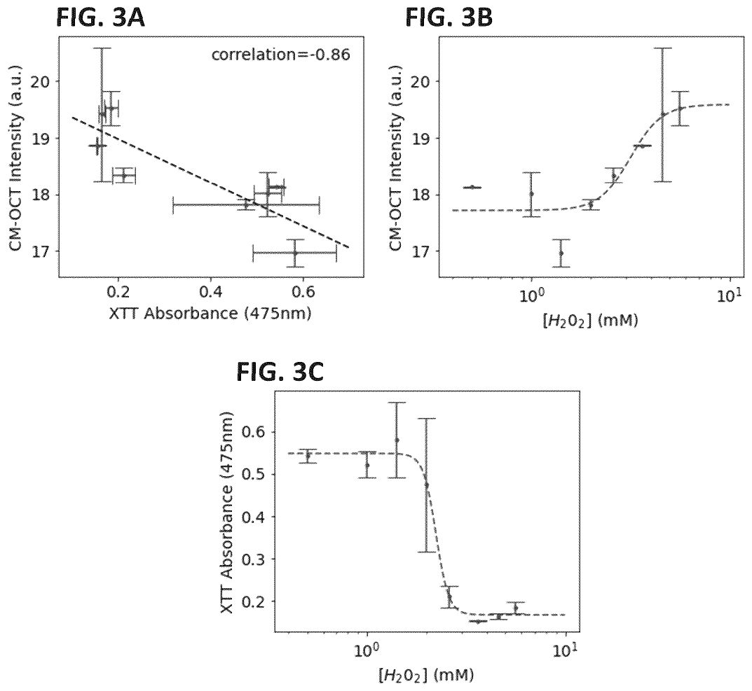

FIG. 3A depicts a comparison of cell viability measures made by a comparative XTT assay absorbance versus measurements made by a CM-OCT system according to one embodiment of the present disclosure. FIG. 3B depicts cell viability measures based on average cell backscatter intensity measured by a CM-OCT system according to one embodiment of the present disclosure. FIG. 3C depicts cell viability measures based on an XTT assay absorbance. Error bars show standard deviation over two separate trials.

As shown by the decrease in XTT absorbance (see FIG. 3C) and an increase in CM-OCT intensity (see FIG. 3B), some experiments showed that cell viability drops dramatically at approximately 2 mM H₂O₂ over 2 hours. The experiment was performed twice and the two measures of viability correlated with a correlation coefficient of ˜0.86 (see FIG. 3A), thereby showing that the cell viability method using CM-OCT intensity is well-correlated with a comparative XTT assay.

### Example 4

In order to test the ability of a CM-OCT system according to some embodiments of the present disclosure to monitor cells over the course of their growth cycle, an experiment was performed by starting a 250 mL culture of Human Embryonic Kidney Cells (HEK293-6E) at 5×10⁵ cells mL⁻¹ and growing the cells in an elliptically shaken (130 RPM) 500 mL Erlenmeyer flask for 160 hours under standard culture conditions (37° C., 5% CO₂). CM-OCT measurements were taken every 30 minutes over the course of the growth. The cells are typically grown for 72 hours before splitting, but the experiment extended the growth to 170 hours in order to observe a decrease in cell viability due to starvation. The experiment was repeated three times with fresh cells each time. The initial cell concentration measurement was computed using an Innovatis Cedex HiRes automated cell counter (“Cedex”). This initial cell concentration measurement was combined with the initial detected objects per second (OPS) in order to convert subsequent OPS measurements into cell concentrations by assuming a linear relationship between OPS and actual cell concentration.

The Cedex is a fully automated, image-based cell analyzer that uses Trypan Blue staining and a high-resolution image scanner to analyze cells and other objects such as aggregates or cell debris. The Cedex uses a proprietary image analysis technique that can distinguish living and dead cells from cell debris, and that can aggregate and calculate cell concentration, viability, aggregation, and morphological parameters, such as cell diameter and compactness.

The embodiment of the CM-OCT system used in this experiment is able to distinguish cells from other objects only by size. Therefore, while Cedex cell concentration measurements can be compared to CM-OCT cell concentration measurements, CM-OCT size measurements are compared to the entire population of objects detected by Cedex.

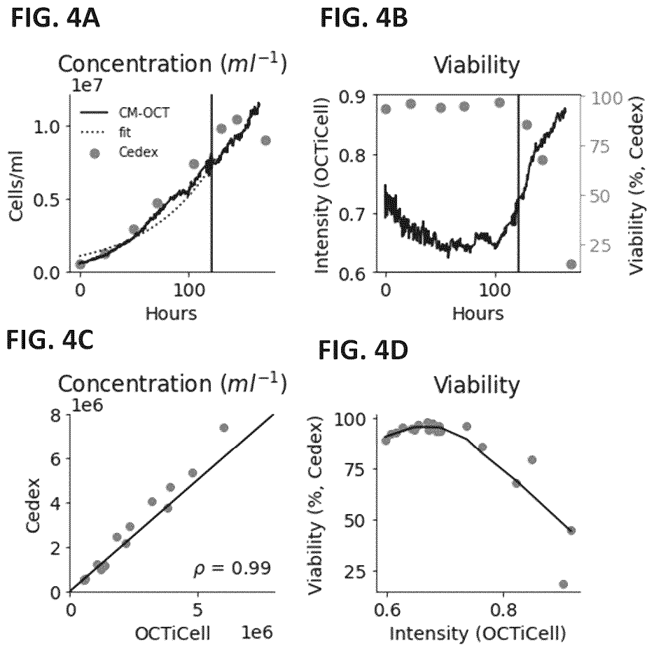

FIG. 4A-D shows data from culture of cells grown over 170 hours in a bioreactor that was monitored by both CM-OCT and a comparative Innovatis Cedex HiRes system. CM-OCT measurements are shown in black and Cedex measurements are shown in grey. The cells were grown to starvation to detect the resulting decrease in viability. The growth experiment was repeated three times. Vertical lines mark the time at which the cell culture viability falls below 90% as determined by backscatter intensity. FIG. 4A Shows cell concentration measurements by CM-OCT and the Cedex system. FIG. 4B Shows the viability and relative intensity of backscattered light as measured by the CM-OCT. FIG. 4C CM-OCT and Cedex measurements of cell concentration taken with cultures with >90% viability in each of the three independent growths. Solid line indicates y=x.

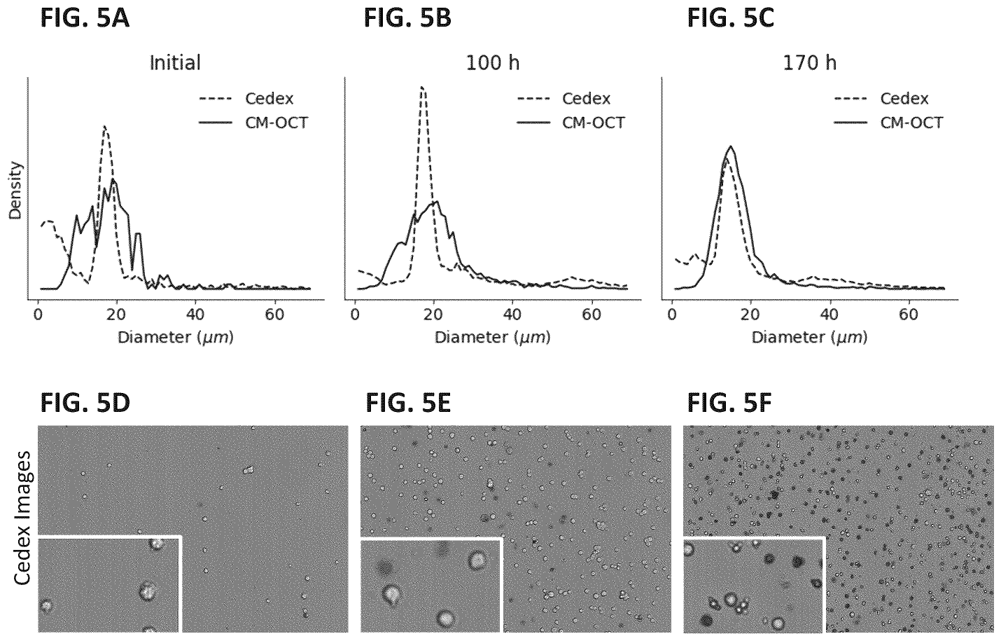

FIG. 5A, FIG. 5B, and FIG. 5C respectively depict the distribution of detected object sizes as measured by the comparative Cedex analyzer and a CM-OCT system according to one embodiment of the present disclosure at the beginning of growth, after 100 hours (as cells are beginning to die), and after 170 hours (when cell viability is low), respectively. FIGS. 5D and 5E depict examples of Cedex images used to measure the object size distribution at the beginning of growth, after 100 hours (as cells are beginning to die), and after 170 hours (when cell viability is low), respectively.

The distribution in detected objects size for CM-OCT gives a wider distribution of sizes for objects in the 10-20 μm range and also has trouble detecting small (<10 μm) and large (>40 μm) objects (see FIG. 5A, FIG. 5B, and FIG. 5C). Cedex estimates of cell viability (trypan blue) decreased dramatically as expected after approximately 100 hours of growth which correlated well with the increase in backscatter intensity measured by CM-OCT (ρ=−0.87; see FIG. 4B and FIG. 4D). As shown in FIGS. 4A and 4C, the concentration measured this way correlated well with cell concentrations as measured by Cedex over the course of the viable growth period (ρ=0.99).

Accordingly, aspects of embodiments of the present disclosure relate to applying OCT to monitor cells growing in agitated bioreactors. By using the motion of the cells through the bioreactor for imaging, embodiments of the present disclosure enable direct in-line quantification of cells and can be used with different kinds of bioreactors by using appropriate probe mounts to align the probe with a window of the bioreactor. In addition, embodiments of the present disclosure can operate while the sample arm fiber is in constant motion, which would be required for an optical monitoring method to image cells in moving bioreactors such as shake-flasks or GE® WAVE Bioreactors. Some aspects of embodiments relate to an optical probe that is small and that does not contain electrical components, thereby allowing probes according to embodiments to be used in medium-scale bioreactors such as the shake flasks demonstrated here, which operate in humid CO₂-filled incubators and for which there are currently no in-line monitoring solutions. Because the system does not require user-interaction after the probe is aligned, the cells can be monitored remotely, continuously, and in real-time (e.g., during cell culture growth).

SPL/spl

1. A system for monitoring states of cells in a bioreactor, the system comprising:
an optical coherence tomography system comprising:
a reference arm coupled to a mirror;
a sample arm coupled to an optical probe configured to emit an imaging beam into the bioreactor, the bioreactor being configured to agitate an aqueous medium;
a light source; and
a spectrometer configured to detect recombined light reflected along the reference arm and the sample arm; and
a controller comprising a processor and memory, the controller being configured to receive spectrometer data from the optical coherence tomography system and to compute statistics of objects detected in the agitated aqueous medium by the optical coherence tomography system.

2. The system of claim 1, wherein the memory stores instructions that, when executed by the processor, cause the processor to:
capture the spectrometer data from the optical coherence tomography system over a capture period;
compute a plurality of 1D images from the spectrometer data, each of the 1D images representing depths of one or more reflective surfaces in the bioreactor from the optical probe along an axis of the imaging beam;
generate a plurality of cell monitoring optical coherence tomography (CM-OCT) frames comprising a plurality of 1D images collected over the capture period; and
compute statistics of the objects detected in the CM-OCT frames.

3. The system of claim 2, wherein the capture period is about 65 ms.

4. The system of claim 2, wherein the memory stores instructions that, when executed by the processor, cause the processor to compute the statistics of the objects detected in the CM-OCT frames by:
detecting and removing a window of the bioreactor from the CM-OCT frames;
detecting the objects in the CM-OCT frames;
computing size and backscatter intensity parameters of each of the objects in the CM-OCT frames, the size parameters comprising a height in a depth dimension and a width in a time dimension of the CM-OCT frames; and
computing the statistics of the objects based on the size and backscatter intensity parameters of the objects.

5. The system of claim 4, wherein computing the size and backscatter intensity parameters of each of the objects comprises a multi-dimensional regression analysis to the object.

6. The system of claim 4, wherein the statistics of the objects comprise a concentration of cells in the bioreactor.

7. The system of claim 4 wherein the plurality of 1D images computed are analyzed using machine learning methods to identify live and dead cells in the bioreactor.

8. The system of claim 4, wherein the memory stores instructions that, when executed by the processor, further cause the processor to determine a set of operating parameter ranges for the statistics based on measurements of other components in the bioreactor.

9. The system of claim 4, wherein the statistics of the objects comprise a viability metric of cells in the bioreactor based on the backscatter intensity parameters of the objects.

10. The system of claim 1, wherein the light source comprises a super-luminescent diode.

11. The system of claim 1, wherein the optical probe comprises a 10× objective.

12. The system of claim 1, wherein the light source has a bandwidth of between 150 and 200 nm.

13. The system of claim 1, wherein the optical probe has a numerical aperture of about 0.064.

14. The system of claim 1, wherein the optical probe comprises an achromatic lens.

15. The system of claim 1, wherein the optical probe is coupled to the bioreactor such that the imaging beam is substantially perpendicular to a wall of the bioreactor.

16. The system of claim 1, wherein the reference arm comprises a variable optical attenuator.

17. A method for monitoring states of cells in a bioreactor using an optical coherence tomography system comprising a processor and memory, the method comprising:
capturing, by the processor, spectrometer data from the optical coherence tomography system over a capture period;
computing, by the processor, a plurality of 1D images from the spectrometer data, each of the 1D images representing depths of one or more reflective surfaces in the bioreactor from an optical probe system along an axis of an imaging beam, wherein the bioreactor is configured to agitate an aqueous medium;
generating, by the processor, a plurality of cell monitoring optical coherence tomography (CM-OCT) frames comprising a plurality of 1D images of the agitated aqueous medium collected over the capture period; and
computing, by the processor, statistics of objects detected in the CM-OCT frames.

18. The method of claim 17, wherein the capture period is between 10 and 100 ms.

19. The method of claim 17, wherein the method of computing the statistics of the objects detected in the CM-OCT frames comprises:
detecting and removing a window of the bioreactor from the CM-OCT frames;
detecting the objects in the CM-OCT frames;
computing size and backscatter intensity parameters of each of the objects in the CM-OCT frames, the size parameters comprising a height in a depth dimension and a width in a time dimension of the CM-OCT frames; and
computing the statistics of the objects based on the size and backscatter intensity parameters of the objects.

20. The method of claim 19, wherein the method for computing the size and backscatter intensity parameters of each of the objects further comprises a multi-dimensional regression analysis to the object.

21. The method of claim 19, wherein the statistics of the objects comprise a concentration of cells in the bioreactor.

22. The method of claim 21, wherein the plurality of 1D images computed are analyzed using machine learning methods to identify live and dead cells in the bioreactor.

23. The method of claim 19, wherein the statistics of the objects comprise a viability metric of cells in the bioreactor based on the backscatter intensity parameters of the objects.

24. The method of claim 17, wherein a reference arm of the optical coherence tomography system comprises an optical arm comprising a variable optical attenuator.

25. A system for monitoring states of cells in a plurality of bioreactors, the system comprising:
an optical coherence tomography system comprising:
a reference arm coupled to a mirror;
a sample arm coupled to a first optical probe configured to emit a first imaging beam into a first bioreactor;
the sample arm coupled to a second optical probe configured to emit a first imaging beam into a second bioreactor, the first bioreactor and the second bioreactor each being configured to agitate, respectively, a first aqueous medium and a second aqueous medium;
an optical switch coupled to the sample arm configured to switch between the first optical probe and the second optical probe;
a light source; and
a spectrometer configured to detect recombined light reflected along the reference arm and the sample arm; and
a controller comprising a processor and memory, the controller being configured to receive spectrometer data from the optical coherence tomography system and to compute statistics of objects detected in the agitated first aqueous medium and in the agitated second aqueous medium by the optical coherence tomography system.
